(19) United States
(12) Patent Application Publication
(10) Pub. No.: US 2008/0214480 A1
(43) Pub. Date: Sep. 4, 2008

(54) Method for Treating Sickle Cell Disease and Sickle Cell Disease Sequalae

(75) Inventors: Chen M. Yu, Mountain Veiw, CA (US); Edgar G. Engleman, Atherton, CA (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: TRF Pharma, Inc., Burlingame, CA (US)

(21) Appl. No.: 11/996,261

(22) PCT Filed: Jul. 21, 2006
(86) PCT No.: PCT/US06/28679

§ 371 Date: Jan. 18, 2008

Related U.S. Application Data
(60) Provisional application No. 60/701,631, filed on Jul. 22, 2005.
(60) Provisional application No. 60/741,839, filed on Dec. 1, 2005.

(51) Int. Cl. A61K 31/7028; A61K 31/715; A61P 7/00

(52) U.S. Cl. 514/25; 514/54

(57) ABSTRACT

The present invention is directed to methods of treating sickle cell disease and its sequelae, including vaso-occlusive crisis. The method comprises administering to a subject in need thereof a pharmaceutical composition comprising an elective amount of a polyanionic polysaccharide, such as pentosan sulfate, sulodexide, or its pharmaceutically acceptable salts thereof. The methods of the present invention are useful in reducing the incidence, severity, or duration of SCD and its sequelae. The compound of the present method can also be used in conjunction with other therapeutic agents useful to treat sickle cell disease thus enhancing the therapeutic elect of reducing the required dosed to treat sickle cell disease.

### FIELD OF THE INVENTION

The present invention relates to methods of treating sickle cell disease and its sequelae with polyanionic polysaccharides. The present invention is exemplified by the treatment of sickle cell disease with pentosan polysulfate.

### BACKGROUND OF THE INVENTION

Sickle cell disease (SCD) is an inherited disorder due to homozygosity for the abnormal hemoglobin, hemoglobin S (HbS). This abnormal hemoglobin S is caused by the substitution of a single base in the gene encoding the human B-globin subunit. Its reach is worldwide, affecting predominantly people of equatorial African descent, although it is found in persons of Mediterranean, Indian, and Middle Eastern lineage. Vaso-occlusive phenomena and hemolysis are clinical hallmarks of SCD. Vaso-occlusion results in recurrent painful episodes (sometimes called sickle cell crisis) and a variety of serious organ system complications among which, infection, acute chest syndrome, stroke, splenic sequestration are among the most debilitating. Vaso-occlusion accounts for 90% of hospitalizations in children with SCD, and it can ultimately lead to life-long disabilities and/or early death.

The pathophysiology of vaso-occlusion is complex and not yet fully understood. Polymerization of deoxygenated hemoglobin S produces sickled cells that cause vaso-occlusion. Abnormal interactions between these poorly deformable sickled cells and the vascular endothelium result in dysregulation of vascular tone, activation of monocytes, upregulation of adhesion molecules and a shift toward a procoagulant state. Current thought suggests that vaso-occlusion is a two-step process. First, deoxygenated sickle cells expressing pro-adhesive molecules adhere to the endothelium to create a nidus of sickled cells. Behind this nidus, sickled cells accumulate behind this blockage to create full blown vaso-occlusion.

The early adhesion of sickled erythrocytes to vascular endothelium appears to be mediated by defined ligand-receptor interactions between endothelial molecules like P-selectin, E-selectin, VCAM-1, thrombospondin, vWf, laminins, and integrins with erythrocytes that express integrin α4β1, CD36, sulfated glycolipids, BCAM/Lu and other pro-adhesion molecules. In addition, abnormal adhesive interactions between leukocytes, platelets, and RBC may also play a role in facilitating nidus formation.

Researchers have tested heparin for application in sickle cell disease. Matsui (Blood. 2002; 15;100:3790-6) studied the effects of heparin on the parameters of rolling velocity and amount of adherent sickle cells. Heparin inhibited in vitro flow adhesion of sickle cells to immobilized P-selectin although only unfractionated and not low molecular weight heparin proved to be effective in this regard. Barabino et al (Blood 1999, 93:1422-1429) demonstrated that dextran sulfate, chondroitin sulfate, and heparin inhibited sickle cell adhesion to HUVEC cells but heparin sulfate did not. Gupta et al (Biochim Biophys Acta 1453:63-73) noted that heparin inhibited sickle erythrocyte adhesion to human endothelial cells in vitro.

Chaplin et al (East Afr Med J. 1989; 66(9):574-84) performed a pilot trial with mini dose heparin in four patients with sickle cell crises. These four patients received 12-month courses of self-administered intravenous minidose heparin and were evaluated weekly or bi-weekly for symptoms and signs of sickle crises. The observations were compared with identical observations during 12 months off heparin (control). All patients had improvement in pain reduction while receiving heparin; 1 moderately, 3 markedly. Pretreatment pain patterns recurred when heparin was discontinued.

Heparin is not suitable for preventing or treating sickle cell disease and sickle vaso-occlusive crisis for a number of reasons. First, heparin is an intravenous drug with virtually no oral bioavailability. Thus, it is not a feasible chronic treatment that a patient could use at home for prophylaxis. Second, heparin has significant, well-known anti-coagulant effects that make it too dangerous either for prophylactic or acute use. Third, heparin has been associated with heparin-induced thrombocytopenia, a severe immune-mediated drug reaction that can occur in any patient exposed by heparin. It is a potentially devastating prothrombotic disease caused by heparin-dependent antibodies that develop either after a patient has been on heparin for 5 or more days or if the patient has had previous heparin exposure. Fourth, the link between cell adhesion and clinical vascular occlusion has not been fully characterized. Thus, despite the publication of the original pilot study by Chaplin in 1989, there have been no additional studies examining the use of heparin for treating vaso-occlusive crisis or treating sequelae of SCD other than pain in over 16 years.

Pentosan polysulfate (PPS) has been studied for use as an anti-cancer compound (Zaslau, Am J Surg. 2004; 188: 589-92; Elliot, Prostate Cancer Prostatic Dis. 2003; 6(2):138-42; Mucha, Oncol. Rep. 2002; 9(6):1385-9; Lush, Ann Oncol. 1996; 7(9):939-44; Schwartsmann, Tumori. 1996; 82:360-3.), drug-delivery carrier, treatment for prion related disease (Doh-ura, Rinsho Shinkeigaku. 2003; 43: 820-2), and prevention of osteoarthritis (Uthman, Postgrad Med J. 2003; 79: 449-53). PPS has also been reported useful in treating urinary tract infections and interstitial cystitis (U.S. Pat. No. 5,180,715), and in combination with an angiostatic steroid, in arresting angiogenesis and capillary, cell or membrane leakage (U.S. Pat. No. 4,820,693).

Fukuda et al (Transpl Int. 2002; 15(1):17-23) examined the level of reperfusion edema in transplanted livers with and without pretreatment with PPS. PPS pre-treated livers showed no reperfusion edema with significantly fewer leukocytes adhering to the vascular wall.

Other research has demonstrated that PPS has anti-adhesive properties that limit adhesion and migration of tumor cells. Schamhart (Urol Res. 1997; 25 Suppl 2:S89-96) demonstrated in vitro that PPS inhibited the growth and migration of tumor cell lines dependent on anchorage (LNCaP and DU145) but had no effect on the growth and migration of cell lines not dependent on anchorage (PC-3). By direct measurements of tumor cell adhesion, Schamhart showed that PPS inhibited tumor cell adhesion, whereas heparin, heparan sulfate, dermatan sulfate, chondroitin-4 sulfate, chondroitin-6 sulfate did not. There is no report as yet showing the effect of PPS on sickle cell adhesion to vascular endothelium.

There is a need of a method for an acute or chronic, orally administered treatment for SCD and its sequelae, in particular, vaso-occlusive crisis. This method should be effective, have no significant adverse effect, and improve patient compliance.

### SUMMARY OF THE INVENTION

The present invention is directed to a method of treating SCD and its SCD sequelae. The method comprises the steps of administering to a subject suffering from such disease a pharmaceutical composition comprising an effective amount of a polyanionic polysaccaride or a pharmaceutically acceptable salt thereof, wherein said polyanionic polysaccaride is selected from the group consisting of pentosan polysulfate, sulodexide, xylan sulfates, dextran sulfates, chitin sulfates, di-, tri-, or oligomers and polymers of iduronic/uronic acids, keratan sulfates, dermatan sulfates, hyaluronic acid, chondroitin sulfate, and the combination thereof.

The invention provides, more particularly, a method for the chronic, prophylactic treatment of vaso-occlusive crisis to reduce the incidence, duration, or severity of vaso-occlusive crisis and acute chest syndrome in SCD patients. Preferred polyanionic polysaccharides useful for this invention are pentosan polysulfate and sulodexide.

The pharmaceutical composition of pentosan polysulfate is administered in the range of 100 mg to 3600 mg per day through a variety of routes of administration, including oral, topical, rectal, injection, or implantation. A preferred route of chronic administration is via oral dosing at a dosing range between 100 mg to 900 mg daily. A preferred route of acute administration is via oral dosing at a dosing range between 300 mg to 1800 mg daily.

The pharmaceutical composition of sulodexide is administered in the range of 100 mg to 3600 mg per day through a variety of routes of administration, including oral, topical, rectal, injection, or implantation. A preferred route of chronic administration is via oral dosing at a dosing range between 200 mg to 400 mg daily. A preferred route of acute administration is via oral dosing at a dosing range between 200 mg to 1800 mg daily.

### DETAILED DESCRIPTION OF THE INVENTION

The present invention provides a method of treating diseases characterized by abnormal adhesion between any combination of erythrocytes, leukocytes, platelets and vascular endothelium. The present invention provides a method of treating a subject suffering from SCD and/or SCD sequelae. The method comprises the steps of administering to a subject in need thereof a pharmaceutical composition comprising an effective amount of a polyanionic polysaccharide or a synthetic polycarboxylic polymer, or a pharmaceutically acceptable salt thereof. The pharmaceutical composition can be administered by either acute or chronic prophylactic administration.

A sickle cell disease is defined herein as homozygous sickle cell disease (hemoglobin SS disease), doubly heterozygous sickle hemoglobin C disease (hemoglobin SC disease) and the sickle β-thalassemias. SCD sequelae refer to any condition or secondary effect of SCD. SCD sequelae include vaso-occlusive crisis; acute chest syndrome; bone complications such as infraction, necrosis, orbital compression syndrome, and arthritis; reproductive complications such as early abortion, intrauterine growth restriction, fetal death, low birth weight, pre-eclampsia, and maternal complications; decreased or stunted growth; priapism; cardiovascular events such as cerebrovascular events and myocardial infarction; and dermatologic complications such as leg ulcers. The pharmaceutical composition is administered in an amount and duration effective to reduce the incidence, severity or duration of such sequelae.

The polyanionic polysaccharides or the synthetic polycarboxylic polymers useful for the present invention include pentosan polysulfate, sulodexide, xylan sulfates, dextran sulfates, chitin sulfates, di-,tri-, or oligomers and polymers of iduronic/uronic acids keratan sulfates, dermatan sulfates, hyaluronic acid, chondroitin sulfate (A, B, C). More than one polyanionic polysaccharides or the synthetic polycarboxylic polymers can be used in combination in the present invention to enhance the therapeutic effects. Preferred compounds are pentosan polysulfate and sulodexide.

Pentosan polysulfate (PPS) is a highly sulfated, semi-synthetic polysaccharide with a molecular weight ranging from about 1,500 to 6,000 Daltons, depending on the mode of isolation. PPS is commercially available as ELMIRON®, Ortho-McNeil. Applicants have discovered that PPS is useful for treating sickle cell disease and its sequelae, by blocking abnormal adhesion among sickle erythrocytes, leukocytes, and vascular endothelium.

PPS and heparin have a number of significant differences in chemical structure, methods of derivation, and physico-chemical properties. Heparin is a sulfated polymer of repeating double sugar monomers, (D)-glucosamine and (D)-glucuronic acid (both 6-carbon hexose sugars), with an amine function on the glucosamine. PPS is a sulfated linear polymer of repeating single monomers of (D)-xylose, a 5-carbon pentose sugar in its pyranose ring form. While heparin rotates plane polarized light in a dextrorotatory direction, PPS rotates light in a levorotatory direction.

While heparin is usually isolated from mammalian tissues such as beef and pork muscles, liver and intestines, PPS is a semi-synthetic compound whose polysaccharide backbone, xylan, is extracted from the bark of the beech tree or other plant sources and then treated with sulfating agents such as chlorosulfonic acid or sulfuryl trichloride and acid. After sulfation, PPS is usually treated with sodium hydroxide to yield the sodium salt.

Unlike heparin, which is digested and cannot pass through gut wall, pentosan polysulfate sodium is orally available. Oral administration provides particular advantages because it allows subjects to take the medication at home, without the need for hospitalization. Thus, oral administration is ideal for prophylactic use by patients outside a hospital setting. Furthermore, since most patients suffering from a vaso-occlusive crisis initially experience symptoms at home for up to 48 hours prior to hospitalization, high dose oral administration at home allows treatment early in the course of a vaso-occlusive crisis when its effects are in general maximal.

PPS provides other advantages when treating SCD. PPS prolongs partial thromboplastin time and has been used to prevent deep venous thrombosis, but it has only about one-fifteenth the anticoagulant potency of heparin (Wardle, J. Int. Med. Res., 20:361-370, 1992). The most severe bleeding event seen in patients with heparin treatment has been rectal hemorrhage. Based on the registration trial data of 2,499 patients for ELMIRON®, only a small portion of patients had an increased prothrombin time, partial thromboplastin time at daily doses between 300 mg-900 mg per day.

There is yet another benefit of oral administration of PPS for the treatment of SCD and SCD sequelae. Administration of PPS by subcutaneous, sublingual, or intramuscular routes, although rare, can be associated with delayed immunoallergic thrombocytopenia, while oral administration is associated with a virtually non-existent rate of delayed immunoallergic thrombocytopenia. This is because oral administration enables liver metabolism after absorption in the gut, which appears to reduce the immunoallergic effect.

Sulodexide (ALFA Wassermann S.p.A Bologna, Italy) is a natural extract from bowel mucosa, which contains a heparin-like substance (80%) and dermatan sulphate (20%). Like PPS, sulodexide differs from heparin in that it has limited anticoagulant efficacy and can be administered orally.

With the advantages over heparin, PPS and sulodexide are useful agents for the treatment of SCD and the reduction of the incidence, duration, and severity of SCD sequelae, particularly vaso-occlusive crisis. PPS and sulodexide reduce sickle cell adhesion to the endothelium and other sickled cells to reduce the extent of vaso-occlusion.

The amount of active compound administered depends on the subject being treated, the severity of the affliction, the manner of administration and the judgment of the prescribing physician. However, an effective dosage is in general in the range of 100 to 3600 mg/day, preferably 100-900 mg/day, which can be administered all at a time or in divided doses such as two, three or four doses. The dosage of these compounds can vary in accordance with the administration route, the age of the patient and the degree of the therapeutic effect desired.

Preferably, the active compound is taken chronically (100-900 mg/day) in oral form in patients with SCD to reduce the incidence, duration, or severity of vaso-occlusive crisis, acute chest syndrome, and other sequelae of the disease. The advantage of an orally dosed medication is that the subject can take the active compound at home rather than in the hospital, which allows treatment prior to the onset of severe symptoms. The invention also describes the use of PPS acutely in a higher dose, in the range of 300-1800 mg/day, to treat the acute onset of vaso-occlusive crisis to reduce the severity or duration of crisis.

The compounds of the present invention can be administered by any of the accepted modes of systemic administration including oral, parenteral, intravenous, intramuscular, and subcutaneous, transdermal, transmucosal, and rectal; with oral administration being preferred.

Any pharmaceutically acceptable mode of administration can be used, including solid, semi-solid, or liquid dosage forms, such as, tablets, suppositories, pills, capsules, powders, granulars, liquids suspensions, injections, or the like, preferably in unit dosage form suitable to single administration of precise dosages, or in sustained or controlled release forms for the prolonged administration of the compound at a predetermined rate. The compositions typically include a conventional pharmaceutical carrier or excipient and the active compound(s) and, in addition, can include other medicinal agents, pharmaceutical agents, carriers, etc. Many examples of such pharmaceutically acceptable vehicles can be found in Remington's Pharmaceutical Sciences (17^{th} edition (1985)) and other standard texts. These preparations can be prepared by any conventional methods.

The carriers useful for these preparations include all organic or inorganic carrier materials that are usually used for the pharmaceutical preparations and are inert to the active ingredient. Examples of the carriers suitable for the preparation of tablets capsules, granules and fine granules are diluents such as lactose, starch, sucrose, D-mannitol, calcium sulfate, or microcrystalline cellulose; disintegrators such as sodium carboxymethylcellulose, modified starch, or calcium carboxymethylcellulose; binders such as methylcellulose, gelatin, acacia, ethylcellulose, hydroxypropylcellulose, or polyvinylpyrrolidone; lubricants such as light anhydrous silicic acid, magnesium stearate, talc, or hydrogenated oil; or the like. When formed into tablets, they can be coated in a conventional manner by using the conventional coating agents such as calcium phosphate, carnauba wax, hydroxypropyl methylcellulose, macrogol, hydroxypropyl methylphthalate, cellulose acetate phthalate, titanium dioxide, sorbitan fatty acid ester, or the like. The preferred carriers for oral administration are those used in the commercial preparation of ELMIRON®.

Examples of carriers suitable for the preparation of syrups are sweetening agents such as sucrose, glucose, fructose, or D-sorbitol; suspending agents such as acacia, tragacanth, sodium carboxymethylcellulose, methylcellulose, sodium alginate, microcrystalline cellulose, or veegum; dispersing agents such as sorbitan fatty acid ester, sodium lauryl sulfate, or polysorbate 80; or the like. When formed into syrups, the conventional flavoring agents, aromatic substances, preservatives, or the like can optionally be added thereto. The syrups can be in the form of dry syrup that is dissolved or suspended before use.

Examples of carriers used for the preparation of suppositories are cacao butter, glycerin saturated fatty acid ester, glycerogelatin, macrogol, or the like. When formed into suppositories, the conventional surface active agents, preservatives or the like can optionally be admixed.

When formed into injections, the compound is dissolved in a suitable solvent for injection, to which can optionally be added the conventional solubilizers, buffering or pH adjusting agents, isotonic agents, preservatives and other suitable substances. The injections can be in the solid dry preparations, which are dissolved before use.

For solid compositions, conventional non-toxic carriers include, for example mannitol, lactose, starch, magnesium stearate, sodium saccharin, talcum, cellulose, glucose, sucrose, magnesium carbonate, and the like can be used. The active compound as defined above can be formulated as suppositories using, for example, polyalkylene glycols such as propylene glycol as a carrier. Liquid pharmaceutically administerable compositions can, for example, be prepared by dissolving, dispersing, etc. an active compound as defined above and optional pharmaceutical adjuvants in a carrier to form a solution or suspension. If desired, the pharmaceutical composition can also contain minor amounts of non-toxic auxiliary pH buffering agents and the like, for example, sodium acetate, sorbitan monolaurate, triethanolamine oleate, etc. Actual methods of preparing such dosage forms are known, or will be apparent to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

The oral pharmaceutical compositions of PPS usually are supplied in white opaque hard gelatin capsules containing 100 mg pentosan polysulfate sodium, microcrystalline cellulose, and magnesium stearate. Such compositions also contain pharmaceutical glaze, synthetic black iron oxide, FD&C Blue No. 2 aluminum lake, FD&C Red No. 40 aluminum lake, FD&C Blue No. 1 aluminum lake, D&C Yellow No. 10 aluminum lake, m-butyl alcohol, propylene glycol, alcohol (SD-3A), lecithin, ethylene glycol, monoethyl ether, and ammonium hydroxide. These compositions optionally contain other therapeutically active compounds.

Dosage forms or compositions contain active ingredient in the range of 0.25 to 95% with the balance made up from non-toxic carrier can be prepared. For oral administration, a pharmaceutically acceptable non-toxic composition is formed by the incorporation of any of the normally employed excipients, and can contain 1%-95% active compound(s), preferably 5%-50%.

Parenteral administration is generally characterized by injection, whether subcutaneously, intramuscularly, or perineurally. Injectables can be prepared in conventional forms, either as liquid solutions, suspensions, or emulsions. In addition, the pharmaceutical compositions can also contain minor amounts of non-toxic substances such as wetting or emulsifying agents, auxiliary pH buffering agents and the like, such as, sodium acetate, sorbitan monolaurate, triethanolamine oleate, etc.

The percentage of active compound(s) contained in such parenteral compositions is highly dependent on the specific nature thereof, as well as the activity of the compound(s) and the needs of the subject.

For delayed release, the compound can be formulated in a pharmaceutical composition, such as in microcapsules formed from biocompatible polymers, nanomilled active compound, or in liposomal carrier systems according to methods known in the art.

For continuous release of active agent, the compound can be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of therapeutics.

The method of the present invention can be used with other therapeutic agents useful to treat SCD, thus enhancing the effects of therapeutic agents and adjunctive agents. Other therapeutic agents include hydroxyurea, inhaled nitric oxide, L-arginine, L-citrulline, anti-coagulants, Gardos channel blockers, and analgesics (including NSAID's, opiates, and acetaminophen.

High doses are sometimes required for some therapeutic agents to achieve levels to effectuate the target response, but high doses often associate with a greater frequency of dose-related adverse effects. Thus, combined use of the pharmaceutical composition of the present invention with therapeutic agents commonly used to treat SCD allows the use of relatively lower doses of other agents, which results in a lower frequency of adverse side effects associated with long-term administration of such agents. Thus, another advantage of the compounds in this invention is to reduce adverse side effects of drugs used to treat SCD, such as tolerance, dependence, alopecia, diarrhea, headache, nausea, rash, dyspepsia, abdominal pain, abnormal liver function, and dizziness.

The following examples further illustrate the present invention. These examples are intended merely to be illustrative of the present invention and are not to be construed as being limiting.

### EXAMPLES

### Example 1

Human SCD patients are enrolled to be studied by a non-invasive videotape of the conjunctival microcirculation. Recently, microvascular characteristics in SCD patients have been quantified using computer-assisted intravital microscopy (CAIM), a novel technology involving intravital video-microscopy coupled with imaging protocols, to study and analyze steady-state and vaso-occlusive crisis (VOC) microvascular characteristics in SCD (Cheung et al, Blood. 2002; 99:3999). During VOC, decreases in vascularity (“blanching”) occur as a result of vasoconstriction and diminished blood flow through the microvessels. Venular diameter and red-cell velocity both decrease significantly as well. The microvascular changes during VOC are transient and revert to steady-state values after crisis resolution. These specific reversals in VOC are used as the basis for this study to evaluate and quantify the effects of PPS on the microcirculation.

Because of the unique shape and form of conjunctival vessels, each vessel is easily identified and located to be re-studied. Each vessel serves as its own reference (control) to evaluate and quantify microvascular changes. The patients are randomized 1:1 after hospitalization for VOC and are given either high dose PPS, sulodexide, or placebo; randomization is conducted off-site and the assignment of PPS, sulodexide, or placebo is blinded to the investigators. CAIM is used to non-invasively videotape and longitudinally quantify the changes in venular diameter and red-cell velocity pre- and post-treatment of PPS, sulodexide or placebo at various time points post treatment. Those in the treatment group show a significant improvement in microvascular flow relative to those in the placebo group.

### Example 2

Human SCD patients are surveyed to establish a baseline level of recurrent vaso-occlusive crisis. Those patients with frequent crisis per annum are enrolled and randomized into groups. One group is given a composition of PPS comprising 100-900 mg/day one time or across divided doses. A second group is given a composition of sulodexide comprising 100-1200 mg/day one time or across divided doses. A third group is given a placebo. These patients are followed for 18 months to assess the difference in rates of vaso-occlusive crisis, acute chest syndrome, days of hospitalization, number of transfusions, use of analgesia, and severity of crisis. A vaso-occlusive crisis is defined as a visit to a medical facility that lasts more than four hours for acute sickling-related pain which is treated with a parenterally administered narcotic (except for facilities in which only orally administered narcotics are used). The measurement of the length of the visit includes all time spent after registration at the medical facility, including the time spent waiting to be seen by a physician. This definition of vaso-occlusive crisis can vary based on different levels of stringency. Sickle related pain is defined as a painful episode in the limbs, vertebral spine, thorax or abdomen, of variable intensity and duration, without other identified cause, preferably recognized by patients and relatives as the characteristic pain caused by the disease. The following clinical trials in sickle cell patients serve as useful examples of this study design (Charache S, NEJM, 1995, 332(20):1317-1322; Alvim R C, Acta Haematologica 2005;113: 228-233).

The treatment groups show a reduction in the rate of crisis, acute chest syndrome, days of hospitalization, number of transfusions, use of analgesia and severity of crisis.

### Example 3

Human SCD patients hospitalized for vaso-occlusive crisis are randomized 1:1 and given either high dose PPS, sulodexide, or placebo. Randomization is conducted off-site and the assignment of treatment or placebo is blinded to the investigator. One group is given a composition of PPS comprising 100-3600 mg/day one time or across divided doses. A second group is given a composition of sulodexide comprising 100-3600 mg/day one time or across divided doses. The third group is given a placebo. These patients are followed to assess the difference in duration of vaso-occlusive crisis, days of hospitalization, use of analgesia, incidence of progression to acute chest syndrome, and severity of crisis as measured by pain scores. Pain scores are tabulated using a ten point visual analog scale. The clinical trial of Poloxmer-188 for acute treatment of vaso-occlusive disease serves as a useful example of this study design (Orringer et al., 2001, JAMA 286(17):2099-2106).

The treatment groups show a reduction in the duration of crisis, days of hospitalization, use of analgesia, incidence of acute chest syndrome, and severity of crisis.

### Example 4

The effects of PPS or sulodexide on sickle cell erythrocyte adhesion are evaluated by in vitro study according to procedures similar to those described in Telen et al. (Blood, 2004, 104:3774); Niihara et al (BMC Blood Disorders, 2005, 5:4); and Barabino et al (Blood, 1999, 93:1422-29). In brief, a variable height flow chamber is used to quantitate the effects of PPS on the adhesion of RBC from sickle cell patients (ssRBC) to endothelial cells. Human umbilical vein endothelial cells (HUVEC) are grown on gelatin coated glass slides until confluent. The HUVEC's are then stimulated with thrombin or TNF-alpha and washed with PBS with Ca⁺² and Mg⁺². Following activation, the endothelial cells are treated with various concentrations of PPS for thirty minutes at 37° C. The endothelial cells are then washed in PBS with Ca⁺² and Mg⁺², mounted onto the variable height flow chamber where the final testing unit is assembled. The flow chamber is mounted on the stage of an inverted phase contrast microscope and the temperature is maintained at 37° C. by a thermoplate (Tokai Hit, Fujinomiya-shi, Japan). ssRBCs are labeled with PKH-26 (Sigma P9691) according to the manufacturer's directions, and resuspended at 0.2% (vol/vol) in 3 ml PBS with Ca⁺² and Mg⁺². The labeled ssRBCs are infused slowly into the flow chamber, are allowed to adhere to the endothelial cell layer, and are then subsequently washed with PBS with Ca⁺² and Mg⁺² for ten minutes at a constant flow rate. Adhesion is quantitated by counting the adherent ssRBCs in a field in 7 different locations along the slide in the direction of the flow. The height of the chamber at each of these 7 locations is measured so that the shear stress can be calculated; note that the typical range seen is between 0.3 to 2.7 dynes/cm². Preparations treated with PPS or sulodexide show a reduction in the percentage of adherent cells versus control preparations.

### Example 5

The effects of PPS or sulodexide on microcirculation are evaluated by in vivo and ex vivo studies according to Kaul et al. (Microcirculation, 2004, 11:153-165); Embury et al. (Blood, 2004,104:3378-3385); Telen (Transfusion Medicine Reviews, 19:1:32-44); Matsui et al. (Blood, 2002, 100:3790-3796); Baez et al. (Flow Properties of Blood and Other Biological Systems, London, UK, Pergamon, 1962, p. 122). In brief, isolated, acutely denervated, artificially perfused rat mesocecum vasculature is modified for the study of erythrocytes. Washed sickle cell erythrocytes, either treated or untreated, are infused into the rat mesocecum vasculature. Peripheral resistance units (PRU) are determined according to Green et al (Handbook of Physiology, Circulation vol. 2, Bethesda, Md., American Physiological Society, 1963, p 935), which provide an indirect measure of cell adhesion. Pressure-flow recovery time (TpF) is determined after the bolus infusion of the blood samples. Direct intravital microscopic observations and simultaneous video recording of the microcirculatory events are performed.

The treated preparations demonstrate a lower PRU and a lower TpF than untreated, control preparations, which indicate reduced adhesion of sickle cell erythrocytes.

### Example 6

15 subjects who are asymptomatic are enrolled in an open label clinical study. Over the next 3 weeks, subjects return to the physician for a physical examination, history, and weekly blood draw to establish baseline levels of surrogate markers and clinical status. The surrogate markers examined include sVCAM-1, C-reactive protein, IL-3, IL-9, Protein C & S, sE-selectin, sP-selectin, and sICAM-1. Sickle cell patients have been found to have higher sVCAM-1 levels than normal subjects (Schnog J B, Ann Hematol. 2003; 82(2):109-13). sVCAM-1 levels also appear to correlate with disease severity as patients with exacerbations of sickle cell disease tend to have even higher sVCAM-1 levels (Saitalkar V S, Am J Hematol. 2004; 76(1):57-60; Kato G J, Br J Haematol. 2005; 130(6):943-53), sVCAM-1 levels also appear to respond to therapeutic treatments for sickle cell enabling it to serve as a viable marker for disease severity in clinical studies (Conran N, Am J Hematol. August 2004; 76(4):343-7.; Sakhalkar V S, Am J Hematol. 2004;76(1):57-60, Liem R I, Am J Hematol. 2004;76(1):19-25; Salch A W, Acta Haematol. 1999;102(1):31-7). Other surrogate markers exhibit similar correlation with sickle cell disease state.

At the end of the 3 week baseline period, subjects begin 3 months of oral treatment on PPS either at a single dose (150-600 mg/day, e.g. 300 mg/day) or multiple doses (50-200 mg three times a day, e.g. 100 mg three times a day). The primary efficacy endpoint would be a comparison of sVCAM 1 at baseline vs either a) the sVCAM-1 level at the 3 months time point or b) the mean of the 3 monthly levels taken during treatment.

Subjects demonstrate a reduced level of sVCAM-1 levels after treatment versus baseline.

### Example 7

15 subjects who are asymptomatic currently but have had frequent crises within the past 12 months are screened in an open label study. Over the next 3 weeks, subjects return to the physician for a physical examination, history, and weekly blood draw to establish baseline levels of surrogate markers and clinical status. The surrogate markers examined include sVCAM-1, C-reactive protein, IL-3, IL-9, Protein C & S, sE-selecting, sP-selectin, and sICAM-1. At the end of the 3 week period, the baseline sVCAM-1 levels are determined and those subjects with elevated sVCAM-1 levels are enrolled into the study to begin 3 months of treatment on PPS at a single dose (150-600 mg/day, e.g. 300 mg/day) or multiple doses (50-200 mg three times a day, e.g. 100 mg three times a day). The primary efficacy endpoint would be a comparison of sVCAM 1 at baseline vs either a) the sVCAM-1 level at the 3 months time point or b) the mean of the 3 monthly levels taken during treatment. The same study is also completed for longer time periods of treatment to maximize separation between treatment group and placebo.

Enrolled subjects demonstrate a reduced level of sVCAM-1 levels after treatment versus baseline.

### Example 8

15 subjects who are asymptomatic are screened and enrolled in a placebo controlled study. Over the next 3 weeks, enrolled subjects return to the physician for a physical examination, history, and weekly blood draw to establish baseline levels of surrogate markers and clinical status. The surrogate markers examined include sVCAM-1, C-reactive protein, IL-3, IL-9, Protein C & S, sE-selectin, sP-selectin, and sICAM-1. At the end of the 3 week period, the baseline sVCAM-1 levels are determined and those subjects with elevated sVCAM-1 levels are randomized to receive placebo or PPS at a single dose (150-600 mg/day, e.g. 300 mg/day) or multiple doses (50-200 mg three times a day, e.g. 100 mg three times a day). The primary efficacy endpoint is a comparison of the change in sVCAM 1 levels from baseline between the treatment and placebo groups. The change in sVCAM-1 is calculated as the difference in baseline sVCAM-1 levels and either a) the sVCAM-1 level at the 3 months time point or b) the mean of the 3 monthly levels taken during treatment. The same study is also completed for longer time periods of treatment to maximize separation between treatment group and placebo.

Treated subjects demonstrate a greater reduction in sVCAM-1 levels versus placebo treated subjects.

### Example 9

15 subjects who are asymptomatic currently but have had frequent crises within the past 12 months are screened and enrolled in a placebo controlled study. Over the next 3 weeks, enrolled subjects return to the physician for a physical examination, history, and weekly blood draw to establish baseline levels of surrogate markers and clinical status. The surrogate markers examined include sVCAM-1, C-reactive protein, IL-3, IL-9, Protein C & S, sE-selecting, sP-selectin, and sICAM-1. At the end of the 3 week period, the baseline sVCAM-1 levels are determined and those subjects with elevated sVCAM-1 levels are randomized to receive 3 months of treatment of either placebo or PPS at a single dose (150-600 mg/day, e.g. 300 mg/day) or multiple doses (50-200 mg three times a day, e.g. 100 mg three times a day). The primary efficacy endpoint is a comparison of the change in sVCAM 1 levels from baseline between the treatment and placebo groups. The change in sVCAM-1 is calculated as the difference in baseline sVCAM-1 levels and either a) the sVCAM-1 level at the 3 months time point or b) the mean of the 3 monthly levels taken during treatment. The same study is also completed for longer time periods of treatment to maximize separation between treatment group and placebo.

Treated subjects demonstrate a greater reduction in sVCAM-1 levels versus placebo treated subjects.

### Example 10

15 subjects who are asymptomatic currently but have had frequent crises within the past 12 months are screened and enrolled in a placebo controlled study. Over the next 3 weeks, enrolled subjects return to the physician for a physical examination, history, and weekly blood draw to establish baseline levels of surrogate markers and clinical status. The surrogate markers examined include sVCAM-1, C-reactive protein, IL-3, IL-9, Protein C & S, sE-selecting, sP-selectin, and sICAM-1. At the end of the 3 week period, the baseline sVCAM-1 levels are determined and those subjects with elevated sVCAM-1 levels are randomized to receive either 3 months of treatment of either placebo or PPS at a single dose (150-600 mg/day, e.g. 300 mg/day) or multiple doses (50-200 mg three times a day, e.g. 100 mg three times a day). The primary efficacy endpoint is a comparison of the change in sVCAM 1 levels from baseline between the treatment and placebo groups. The change in sVCAM-1 is calculated as the difference in baseline sVCAM-1 levels and either a) the sVCAM-1 level at the 3 months time point or b) the mean of the 3 monthly levels taken during treatment. The same study is also completed for longer time periods of treatment to maximize separation between treatment group and placebo.

Treated subjects demonstrate a greater reduction in sVCAM-1 levels versus placebo treated subjects.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications can be made therein without departing from the scope of the present invention as set forth in the claims. To particularly point out and distinctly claim the subject matter regarded as invention, the following claims conclude the specification.

What is claimed is:

1. A method for treating SCD sequelae comprising the steps of administering to a subject in need thereof an effective amount of a compound or a pharmaceutically acceptable salt thereof, wherein said compound is selected from the group consisting of pentosan polysulfate, sulodexide, xylan sulfates, dextran sulfates, chitin sulfates, di-, tri-, or oligomers and polymers of iduronic/uronic acids, keratan sulfates, hyaluronic acid, and combination thereof.

2. The method according to claim 1, wherein said SCD sequela is vaso-occlusive crisis And said compound is administered in an amount and duration effective to reduce the incidence, severity or duration of vaso-occlusive crisis.

3. The method according to claim 1, wherein said SCD sequela is acute chest syndrome and said compound is administered in an amount and duration effective to reduce the incidence, severity or duration of acute chest syndrome.

4. The method according to claim 1, wherein said SCD sequela is a bone complication selected from the group consisting of infarction, necrosis, or orbital compression syndrome, and said compound is administered in an amount and duration effective to reduce the incidence, severity or duration of such sequela.

5. The method according to claim 1, wherein said SCD sequela is a reproductive complication selected from the group consisting of early abortion, intrauterine growth restriction, fetal death, low birth weight, pre-eclampsia, and maternal complications, and said compound is administered in an amount and duration effective to reduce the incidence, severity or duration of such sequela.

6. The method according to claim 1, wherein said SCD sequela is decreased or stunted growth, and compound is administered in an amount and duration effective to reduce the severity of stunted growth.

7. The method according to claim 1, wherein said SCD sequela is priapism and said compound is administered in an amount and duration effective to reduce the incidence, duration or severity of priapism.

8. The method according to claim 1, wherein said SCD sequelae are-cerebrovascular events, and said compound is administered in an amount and duration effective to reduce the incidence, duration or severity of such events.

9. The method according to claim 1, wherein said SCD sequelae are dermatologic complications, and said compound is administered in an amount and duration effective to reduce the incidence, severity or duration of such complications.

10. The method according to any one of claims 1-9, wherein said compound is pentosan polysulfate.

11. The method according to claim 10, wherein said effective amount is in the range of about 100 to about 3600 mg/day given once or across divided doses of two, three or four doses.

12. The method according to claim 11, wherein said effective amount is in the range of about 300 to about 900 mg/day.

13. The method according to any one of claims 1-9, wherein said compound is sulodexide.

14. The method according to claim 13, wherein said effective amount is in the range of about 100 to about 3600 mg/day given once or across divided doses of two, three or four doses.

15. The method according to claim 14, wherein said effective amount is in the range of about 200 to about 1800 mg/day.

16. The method according to any one of claims 1-9, wherein said compound is administered orally to said subject.

17. The method according to any one of claims 1-9, wherein said compound is administered by acute administration at 300-1800 mg daily.

18. The method according to any one of claims 1-9, wherein said compound is administered by chronic administration at 100-900 mg daily.

19. The method according to any one of claims 1-9, wherein said compound is administered in combination with another therapeutic agent useful to treat SCD sequelae.
